SEQUENCE LISTING (6 sequences)

SEQ ID NO: 1 - protein, 68 aa, Homo sapiens
MTCTDQKSHSQRALGTQTPALQGPQLLNTDPSSKETRPPHVNPDRLCHMEPANHFWHAGDLQAMISKE

SEQ ID NO: 2 - protein, 17 aa, Homo sapiens
MASRRHAGDLQAMISKE

SEQ ID NO: 3 - protein, 13 aa, Homo sapiens
MDWGTLQTILGGV

SEQ ID NO: 4 - protein, 55 aa, Homo sapiens
MDWGTLQTILGGVNKHSTSIGKIWLTVLFIFRIMILVVAAKEVWGDEQADFVCNT

SEQ ID NO: 5 - DNA, 60 bp, Homo sapiens
ctcaaggagaaggcggcgccagactggccgagtaccacgccaaggccaccgagcatctga

SEQ ID NO: 6 - DNA, 66 bp, Homo sapiens
ctcaaggagaacggcggcggcagactggccgagtaccacgccaaggccaccgagcatctgcagtga

(19) United States
(12) Patent Application Publication
(10) Pub. No.: US 2008/0003693 A1
(43) Pub. Date: Jan. 3, 2008

(54) METHODS AND MATERIALS FOR DETECTING FRAMESHIFT MUTATIONS

(75) Inventor: Anthony R. Torres, Centerville, UT (US)

Correspondence Address: BRINKS HOFER GILSON & LIONE;UTAH OFFICE, 405 South Main Street, Suite 800, SALT LAKE CITY, UT, 84111-3400, US

(73) Assignee: Great Basin Scientific, Inc.

(21) Appl. No.: 11/761,782

(22) Filed: Jun. 12, 2007

Related U.S. Application Data
(60) Provisional application No. 60/804,482, filed on Jun. 12, 2006.

(51) Int. Cl. G01N 33/53; C07K 16/00

(52) U.S. Cl. 436/501; 530/387.1; 530/388.1

(57) ABSTRACT

The invention relates to methods and materials for detecting in a biological sample the presence or absence of a target protein having a frameshift mutation that results in missense amino acid sequence downstream of the frameshift mutation, comprising combining with the biological sample a binding ligand that is capable of specifically binding the missense amino acid sequence, and then determining whether the binding ligand binds to the missense amino acid sequence. Binding of the ligand to the missense amino acid sequence is indicative of the presence of the frameshift mutation.

### CROSS REFERENCE TO RELATED APPLICATION

This application claims the benefit under 35 U.S.C. § 119(e) of U.S. Provisional Patent Application No. 60/804,482, filed Jun. 12, 2006, which is incorporated, in its entirety, by this reference.

### FIELD OF THE INVENTION

The present invention relates generally to the field of protein identification and characterization, and more particularly to methods and materials for identifying and characterizing proteins encoded by genes having mutations.

### BACKGROUND

A wide variety of biological research and clinical techniques utilize synthetic nucleic acid or other nucleobase polymer probes and primers for the detection, quantification, and characterization of the genetic basis of inherited and infectious diseases. Such techniques typically rely upon hybridization of the nucleic acid probes and primers to complementary regions of DNA or RNA that characterize the disease. Nucleic acid or other nucleobase polymer probes have long been used clinically to analyze samples for the presence of nucleic acid from infectious agents, such as bacteria, fungi, virus or other organisms, and in examining genetically-based diseases.

Protein based assays are also commonly used to identify and characterize diseases associated with genetic mutations that alter protein function. Generally, protein based assays utilize a binding ligand, such as an antibody, that specifically binds to the protein of interest to detect the presence or absence of the protein.

Some protein molecules, however, are characterized by polymorphic variation, which are more difficult to detect and characterize. Accordingly, there is a need for improved methods and reagents for detection, quantification and characterization of proteins altered by genetic mutations.

### SUMMARY OF THE INVENTION

The present invention relates to improved methods and reagents for detection, quantification and characterization of nucleic acid templates having polymorphic variation. More particularly, the present invention relates to methods for using binding ligands that specifically bind target protein epitopes characteristic of genetic mutations.

In one aspect, the present invention relates to a method for detecting the presence or absence of missense amino acid sequence downstream of a position corresponding to a frameshift mutation using a binding ligand, such as an antibody, that is specific for the missense amino acid sequence downstream of and resulting from the frameshift mutation. Thus, in some embodiments, the methods of the invention may comprise

(a) providing a biological sample containing a target protein encoded by a gene characterized by a missense frameshift mutation, wherein a gene having the frameshift mutation encodes a target protein comprising missense amino acid sequence downstream of the position corresponding to the frameshift mutation and a gene not having the frameshift mutation encodes a target protein comprising wild-type amino acid sequence downstream of the position corresponding to the frameshift mutation;

(b) combining with the biological sample a binding ligand capable of specifically binding the missense amino acid sequence; and

(c) determining whether the binding ligand binds to the missense amino acid sequence, wherein binding of the ligand to the missense amino acid sequence is indicative of the presence of the frameshift mutation, and the absence of binding of the ligand to the missense amino acid sequence is indicative of the absence of the frameshift mutation.

In another aspect, the present invention is directed to methods for detecting the presence or absence of a target protein having a frameshift mutation in a biological sample by determining whether a binding ligand, such as an antibody, binds to the wild-type amino acid sequence of a target protein that is downstream of the position corresponding to the frameshift mutation. In some embodiments, the method comprises

(a) providing a biological sample containing a target protein encoded by a gene characterized by a missense frameshift mutation, wherein a gene having the frameshift mutation encodes a target protein comprising missense amino acid sequence downstream of the position corresponding to the frameshift mutation, and wherein a gene not having the frameshift mutation encodes a target protein comprising wild-type amino acid sequence downstream of the position corresponding to the frameshift mutation;

(b) combining with the biological sample a binding ligand capable of specifically binding the wild-type amino acid sequence; and

(c) determining whether the binding ligand binds to the wild-type amino acid sequence, wherein binding of the ligand to the wild-type amino acid sequence is indicative of the absence of the missense amino acid sequence, and the absence of binding of the ligand to the wild-type amino acid sequence is indicative of the presence of the missense amino acid sequence.

In another aspect, the present invention relates to a method for detecting the presence or absence of a target protein in a biological sample using a binding ligand that specifically binds to the wild-type amino acid sequence. In one embodiment, the invention is directed to a method for detecting the presence or absence of a target protein in a biological sample, comprising:

(a) providing a biological sample containing a target protein encoded by a gene characterized by a frameshift mutation, wherein a gene having the frameshift mutation encodes a target protein comprising missense amino acid sequence downstream of the position corresponding to the frameshift mutation, and wherein a gene not having the frameshift mutation encodes a target protein comprising wild-type amino acid sequence downstream of the position corresponding to the frameshift mutation;

(b) combining with the biological sample a binding ligand capable of specifically binding the wild-type amino acid sequence; and

(c) determining whether the binding ligand binds to the wild-type amino acid sequence, wherein binding of the ligand to the wild-type amino acid sequence is indicative of the absence of the missense amino acid sequence, and the absence of binding of the ligand to the wild-type amino acid sequence is indicative of the presence of the missense amino acid sequence.

In another aspect, the present invention is directed to methods for detecting the presence or absence of a nonsense frameshift mutation (i.e., a frameshift mutation that encodes a target protein that is truncated at the position of the frameshift mutation). In one particular embodiment, the invention is directed to methods for detecting the presence or absence of a target protein in a biological sample, comprising:

(a) providing a biological sample containing a target protein encoded by a gene characterized by a nonsense frameshift mutation, wherein a gene having the nonsense frameshift mutation encodes a target protein truncated at the position of the frameshift mutation, and wherein a gene not having the nonsense frameshift mutation encodes a target protein comprising wild-type amino acid sequence downstream of the position corresponding to the frameshift mutation;

(b) combining with the biological sample a binding ligand capable of specifically binding the wild-type amino acid sequence; and

(c) determining whether the binding ligand binds to the wild-type amino acid sequence, wherein binding of the ligand to the wild-type amino acid sequence is indicative of the absence of the nonsense mutation, and the absence of binding of the ligand to the wild-type amino acid sequence is indicative of the presence of the nonsense mutation.

In yet another aspect, the present invention is directed to methods for detecting the presence or absence of a protein encoded by a gene having a splice-site mutation (i.e., a mutation characterized by an internal deletion of a portion of sequence, resulting in a modified splice-site between two wild-type regions). In one particular embodiment, the invention is directed to methods for detecting the presence or absence of a target protein in a biological sample, comprising:

(a) providing a biological sample containing a target protein encoded by a gene characterized by a splice-site mutation, wherein a gene having the splice-site mutation encodes a target protein comprising wild-type sequence having a modified splice-site amino acid sequence and a gene not having the splice-site mutation encodes a target protein comprising normal wild-type amino acid sequence;

(b) combining with the biological sample a binding ligand capable of specifically binding the splice-site amino acid sequence; and

(c) determining whether the binding ligand binds to the splice-site amino acid sequence, wherein binding of the ligand to the splice-site amino acid sequence is indicative of the presence of the splice-site mutation, and the absence of binding of the ligand to the splice-site amino acid sequence is indicative of the absence of the splice-site mutation.

In some embodiments, the methods of the present invention contemplate also use of a second binding ligand specific for the wild-type amino acid sequence encoded by a wild-type gene (i.e., a gene that does not have the frameshift mutation) downstream of the position of the frameshift mutation. A second binding ligand may be useful as a control for verifying the presence or absence of a protein encoded by the wild-type gene. Thus, the methods of the invention may further comprise combining with the biological sample a second binding ligand capable of specifically binding the wild-type amino acid sequence, and determining whether the second binding ligand binds to the wild-type amino acid sequence, wherein binding of the second binding ligand to the wild-type amino acid sequence is indicative of the presence of the wild-type gene.

In other embodiments, the methods of the invention also contemplate use of a control binding ligand specific for a region of the target protein unaffected by the frameshift mutation (e.g., upstream of the position of the frameshift mutation), together with a binding ligand specific for the missense amino acid sequence (or specific for the wild-type sequence in the absence of the frameshift mutation), to confirm the presence of the target protein. Thus, the methods of the invention may further comprise combining with the biological sample a control binding ligand capable of specifically binding the target protein upstream of the position corresponding to the frameshift mutation, and determining whether the control binding ligand binds to the target protein, wherein binding of the control binding ligand to target protein is indicative of the presence of the target protein in the sample.

In yet another aspect, the present invention relates to methods for detecting the presence or absence of both the missense amino acid sequence and the wild-type amino acid sequence. In one embodiment, the methods of the invention comprise combining with the biological sample a control binding ligand capable of specifically binding the target protein upstream of the position corresponding to the frameshift mutation; and determining whether the control binding ligand binds to the target protein, wherein binding of the control binding ligand to target protein is indicative of the presence of the target protein in the sample.

In yet another aspect, the present invention is also directed to antibodies, such as monoclonal antibodies, capable of binding capable of specifically binding a missense amino acid sequence of a target protein, wherein the missense amino acid sequence is downstream of a position corresponding to a frameshift mutation of a gene encoding the target protein. In still another aspect, the present invention is directed to antibodies capable of specifically binding a splice-site amino acid sequence of a target protein.

In still another aspect, the present invention is directed to kits comprising a binding ligand capable of specifically binding a missense amino acid sequence of a target protein, wherein the missense amino acid sequence is downstream of a position corresponding to a frameshift mutation of a gene encoding the target protein.

### DETAILED DESCRIPTION OF THE INVENTION

Units, prefixes, and symbols may be denoted in their SI accepted form. Unless otherwise indicated, nucleic acids are written left to right in 5′ to 3′ orientation; amino acid sequences are written left to right in amino to carboxy orientation. Numeric ranges recited herein are inclusive of the numbers defining the range and include and are supportive of each integer within the defined range. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes. Unless otherwise noted, the terms “a” or “an” are to be construed as meaning “at least one of”. The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. In the case of any amino acid or nucleic sequence discrepancy within the application, the figures control.

Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

Definitions

“Antibody” includes both glycosylated and non-glycosylated immunoglobulins of any isotype or subclass or combination thereof, including human (including CDR-grafted antibodies), humanized, chimeric, multi-specific, monoclonal, polyclonal, and oligomers thereof, irrespective of whether such antibodies are produced, in whole or in part, via immunization, through recombinant technology, by way of in vitro synthetic means, or otherwise. Thus, the term “antibody” includes those that are prepared, expressed, created or isolated by recombinant means, such as (a) antibodies isolated from an animal (e.g., a mouse) that is transgenic for human immunoglobulin genes or a hybridoma prepared therefrom, (b) antibodies isolated from a host cell transfected to express the antibody, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences. Such antibodies have variable and constant regions derived from germline immunoglobulin sequences of two distinct species of animals. In certain embodiments, however, such antibodies can be subjected to in vitro mutagenesis (or, when an animal transgenic for human immunoglobulin sequences is used, in vivo somatic mutagenesis) and thus the amino acid sequences of the VH and VL regions of the antibodies are sequences that, while derived from and related to the germline VH and VL sequences of a particular species (e.g., human), may not naturally exist within that species' antibody germline repertoire in vivo.

A whole antibody is a glycoprotein comprising at least two heavy (H) chains and two light (L) chains inter-connected by disulfide bonds, or an antigen-binding region thereof. Each heavy chain is comprised of a heavy chain variable region (abbreviated herein as VH) and a heavy chain constant region, comprised of three domains (abbreviated herein as CH1, CH2 and CH3). Each light chain is comprised of a light chain variable region (abbreviated herein as VL) and a light chain constant region, comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An amino acid sequence which is substantially the same as a heavy or light chain CDR exhibits a considerable amount or extent of sequence identity when compared to a reference sequence and contributes favorably to specific binding of an antigen bound specifically by an antibody having the reference sequence. Such identity is definitively known or recognizable as representing the amino acid sequence of the particular human monoclonal antibody. Substantially the same heavy and light chain CDR amino acid sequence can have, for example, minor modifications or conservative substitutions of amino acids so long as the ability to bind a particular antigen is maintained. The term “human monoclonal antibody” is intended to include a monoclonal antibody with substantially human CDR amino acid sequences produced, for example, by recombinant methods, by lymphocytes or by hybridoma cells.

“Monoclonal antibody” or “monoclonal antibody composition” as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

“Binding ligand” means any molecule that specifically binds a particular epitope of a protein molecule. Binding ligands include, for example, antibody molecules, receptor molecules, and small-molecule compounds that binding to a specified epitope.

“Characterized by,” as used in the context of a gene “characterized by” a mutation, such as a frameshift mutation, a missense mutation or a nonsense mutation, means that the gene is known to exist in different polymorphic forms within a population of relevant subjects, with some polymorphic forms having the mutation and other polymorphic forms not having the mutation. A given subject may have a single polymorphic form of a gene, or may have multiple polymorphic forms. As used herein, the term “characterized by,” in reference to a particular type of mutation of a protein in a biological sample, does not imply that the biological sample actually has the protein or the mutation associated with the protein; it is, therefore, understood that the methods described herein for detecting the presence or absence of a target protein in a biological sample “containing” a target protein encoded by a gene characterized by a missense frameshift mutation include methods that fail to detect the presence of a mutation in the target protein due to the absence of the mutation in the subject (even though the protein is present) or the absence of the protein altogether.

“Characteristic of,” as used in the context of a modification of the target protein that is “characteristic of” the frameshift mutation, means that the modification of the target protein is indicative of the frameshift mutation in the corresponding gene encoding the protein, and that the mutation in the gene can be detected, identified or inferred by characterizing the presence or absence of the corresponding mutation in the protein.

“Insertion mutation” is a mutation that results in the insertion of one or more nucleic acid bases in a gene.

“Deletion mutation” means a mutation that results in the deletion of one or more nucleic acids bases in a gene.

“Frameshift mutation” means a mutation that results in the insertion or deletion nucleic acid bases in a gene that causes a shift or alteration in the translation reading frame of the gene to result in a modification of at least one codon, resulting in a stop codon or missense amino acid sequence downstream of the mutation. A mutation that causes a shift in the reading frame of a gene results in a protein translation product having amino acid sequence that is not present in the natural protein (missense amino acid sequence) and/or a stop codon at the site of the mutation or downstream of the mutation, resulting in premature termination of translation of the gene. A reading frame consists of groups of 3 bases that each code for one amino acid. A frameshift mutation modifies or shifts the grouping of these bases and changes the three-base code of the correct amino acid for a different amino acid. The resulting protein is usually, but not always, nonfunctional. A frameshift can be caused by an insertion mutation that adds additional nucleotides or a deletion mutation that removes nucleotides. A “frameshift mutation” may also be caused by the deletion of partial codons. For example, the deletion of a fragment that begins with the partial removal of 2 nucleic acids of one codon, and ends with the partial removal of 1 nucleic acid of another codon, may result in the remaining 1 codon at the beginning and 2 codons at the end forming a new codon that encodes a different polymorphic amino acid not present in the wild-type amino acid sequence, even though the downstream amino acids following the polymorphic amino acid remain in the original reading frame and encode an amino acid sequence that matches the wild-type amino acid sequence. Although such a deletion of partial codons results in only a single amino acid change, such a deletion is still considered, for purposes of the present invention, to be a “frameshift mutation,” with the resulting polymorphic amino acid also considered to be a “missense amino acid sequence,” as defined below.

“Frameshift protein product” means a protein that is encoded by a gene having a frameshift mutation.

“Indicative” means determinative of or consistent with.

“Missense amino acid sequence” means amino acid sequence that corresponds to the nucleotide sequence of gene in an incorrect reading frame. Missense amino acid sequence results following a frameshift mutation that shifts or alters the reading frame of a gene to code for different amino acids, resulting in a protein translation product having amino acid sequence that is different from that encoded by the correct reading frame of the gene.

“Missense mutation” means a mutation that inserts or deletes 1 or 2 bases (or any number of bases that is not a multiple of 3) and shifts the reading frame of a gene so as to encode a protein having an altered amino acid sequence downstream of the position corresponding to the gene mutation.

“Modified splice-site amino acid sequence” means amino acid sequence that results from the internal deletion of amino acid sequence in a protein, resulting in a protein having two wild-type sequences spliced together to form a modified splice-site.

“Non-sense mutation” means a mutation that introduces a stop codon (TAA, TAG, or TGA) at the site of the mutation, signaling the end of a protein-coding sequence and resulting in the premature termination of translation of mRNA into a protein molecule.

“Specifically binds” and “specific binding” mean that a compound preferentially or selectively recognizes and binds mature, full-length or partial-length epitope of a protein, or an ortholog thereof, such that its affinity (as determined by, e.g., Affinity ELISA or BIAcore assays as described herein) or its neutralization capability (as determined by e.g., Neutralization ELISA assays described herein, or similar assays) is at least 10 times as great, but optionally 50 times as great, 100, 250 or 500 times as great, or even at least 1000 times as great as the affinity or neutralization capability of the same for any other polypeptide, wherein the peptide portion of the peptibody is first fused to a human Fc moiety for evaluation in such assay. Typically, the antibody binds with an affinity of at least about 1×10⁷ M⁻¹, and binds to the predetermined antigen with an affinity that is at least two-fold greater than its affinity for binding to a non-specific antigen (e.g., BSA, casein) other than the predetermined antigen or a closely-related antigen. As used herein, an antibody “recognizing” or “specific for” an antigen is considered equivalent to “binding specifically” to an antigen. An antibody that specifically binds to a specified epitope, isoform or variant of a particular target protein or a particular region of a target protein may, however, still have cross-reactivity to other related antigens, e.g., from other species (e.g., species homologs) and still be considered to “specifically bind” the specified epitope.

“Upstream,” as used to describe the position of an amino acid or region of amino acids in a protein relative to another amino acid, means the amino-terminal or N-terminal direction. As used to describe the position of a nucleotide or region of nucleotides, “upstream” means the 5′ direction.

“Downstream,” as used to describe the position of an amino acid or region of amino acids in a protein relative to another amino acid, means the carboxy-terminal or C-terminal direction. When describing the effect of a genetic mutation on the “downstream” amino acid sequence of the corresponding protein encoded by the gene, it is understood that the “downstream” amino acid sequences affected include the amino acid encoded by the codon of the nucleotide that is modified. also includes the meaning of “at” (for example, a mutation resulting in a change in the third nucleotide of a codon would result in change in the amino acid encoded “at” that position, as well as the amino acids downstream of that position). As used to describe the position of a nucleotide or region of nucleotides, “downstream” means the 3′ direction.

“Splice variant” means a mutation that deletes normal wild type protein sequences by apparent splicing reactions and joins two segments of the wild type protein not normally joined. This process creases a novel “fusion” peptide region which can be selectively targeted by detection using a peptide binding molecule that specifically recognizes the fusion peptide region of the truncated protein.

“Stop codon mutation” means a point mutation, a deletion, or an insertion that creates a stop codon within a coding sequence of a wild type gene. Stop codon mutations will encode a protein product that is truncated at a position corresponding to the mutation in the gene, resulting in wild type amino acid sequence up to the point of the mutation where the protein is prematurely truncated. Detection of such truncated proteins normally would use two peptide specific binding molecules, one binding molecule specific for sequences present in both wild type and truncated proteins and one binding molecule specific for sequences present only in the non-truncated wild type protein.

“Wild-type amino acid sequence” means amino acid sequence that corresponds to the consensus or most prevalent (within a population of individuals) nucleotide sequence of a gene in its correct reading frame.

“Allele” means one of multiple alternate forms of a polynucleotide template having a particular nucleobase at a polymorphic site. The term “allele” is commonly used to refer to one of two alternate forms of a gene that have a common locus on homologous chromosomes (within a single organism, or among different organisms within a common species) and may be responsible for alternative traits. As used herein, the term “allele” is also used to refer to a particular polymorphic variant (nucleobase) at a polymorphic site of polynucleotide template. It is understood that the term “allele” may be used in reference to alternate forms of any type of polynucleotide template, including synthetic or recombinant polynucleotide templates, as well as natural polynucleotide templates (genes) derived from a natural source.

“Complementary” means that a nucleobase of a polynucleotide is capable of hybridizing to a corresponding nucleobase in a different polynucleotide. As used herein, the term “complementary” is not limited to canonical Watson-Crick base pairs with A/T, G/C and U/A. Thus, nucleobase pairs may be considered to be “complementary” if one or both of the nucleobases is a nucleobase other than A, G, C, or T, such as a universal or degenerate nucleobase. A degenerate or universal nucleobase that is “complementary” to two or more corresponding nucleobases is considered to hybridize equivalently to the two or more corresponding nucleobases. The term “complementary” also refers to antiparallel strands of polynucleotides (as opposed to a single nucleobase pair) that are capable of hybridizing. For example, the sequence 5′-AGTTC-3′ is complementary to the sequence 5′-GAACT-3′. The term “complementary” is sometimes used interchangeably with “antisense.” Thus, degenerate nucleobase oligomers are said to hybridize to a corresponding multi-allelic polynucleotide template. The term “complementary.” as used in reference to two nucleotide sequences or two nucleobases, implies that the nucleotides sequences or nucleobases are “corresponding.”

“Corresponding” means, as between two nucleotide sequences or two nucleobases within a sequence, having the same or nearly the same relationship with respect to position and complementarity, or having the same or nearly the same relationship with respect to structure, function, or genetic coding (for example, as between a gene and the “corresponding” protein encoded by the gene). For example, a nucleotide sequence “corresponds” to region of a polynucleotide template if the two sequences are complementary or have portions that are complementary. Similarly, a nucleobase of an oligomer “corresponds” to a nucleobase of a polynucleotide template when the two nucleobases occupy a position such that when the oligomer and the polynucleotide hybridize the two nucleobases pair opposite each other. The term “corresponding” is generally used herein in reference to the positional relationship between two polynucleotide sequences or two nucleobases. The term “corresponding” does not imply complementarity; thus, corresponding nucleobases may be complementary, or may be non-complementary.

“Nucleic acid” is a nucleobase polymer having a backbone formed from nucleotides, or nucleotide analogs. “Nucleic acid” and “polynucleotide” are considered to be equivalent and interchangeable, and refer to polymers of nucleic acid bases comprising any of a group of complex compounds composed of purines, pyrimidines, carbohydrates, and phosphoric acid. Nucleic acids are commonly in the form of DNA or RNA. The term “nucleic acid” includes polynucleotides of genomic DNA or RNA, cDNA, semisynthetic, or synthetic origin. Nucleic acids may also substitute standard nucleotide bases with nucleotide isoform analogs, including, but not limited to iso-C and iso-G bases, which may hybridize more or less permissibly than standard bases, and which will preferentially hybridize with complementary isoform analog bases. Many such isoform bases are described, for example, at www.idtdna.com. The nucleotides adenosine, cytosine, guanine and thymine are represented by their one-letter codes A, C, G, and T respectively. In representations of degenerate primers or mixture of different strands having mutations in one or several positions, the symbol R refers to either G or A, the symbol Y refers to either T/U or C, the symbol M refers to either A or C, the symbol K refers to either G or T/U, the symbol S refers to G or C, the symbol W refers to either A or T/U, the symbol B refers to “not A”, the symbol D refers to “not C”, the symbol H refers to “not G”, the symbol V refers to “not T/U” and the symbol N refers to any nucleotide.

“Nucleotide” refers to a phosphate ester of a nucleoside, as a monomer unit or within a polynucleotide polymer. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g., alpha.-thio-nucleotide 5′-triphosphates. For a review of polynucleotide and nucleic acid chemistry, see Shabarova, Z. and Bogdanov, A. Advanced Organic Chemistry of Nucleic Acids, VCH, New York, 1994.

“Polymorphic site” means a base position of a polynucleotide characterized by polymorphic variation in the type of nucleobase.

“Polynucleotide” and “oligonucleotide” are used interchangeably and mean single-stranded and double-stranded polymers of nucleotide monomers, including 2′-deoxyribonucleotides (DNA) and ribonucleotides (RNA) linked by internucleotide phosphodiester bond linkages, e.g., 3′-5′ and 2′-5′, inverted linkages, e.g., 3′-3′ and 5′-5′, branched structures, or internucleotide analogs. A “polynucleotide sequence” refers to the sequence of nucleotide monomers along the polymer. “Polynucleotides” are not limited to any particular length of nucleotide sequence, as the term “polynucleotides” encompasses polymeric forms of nucleotides of any length. Polynucleotides that range in size from about 5 to about 40 monomeric units are typically referred to in the art as oligonucleotides. Polynucleotides that are several thousands or more monomeric nucleotide units in length are typically referred to as nucleic acids. Polynucleotides can be linear, branched linear, or circular molecules. Polynucleotides also have associated counter ions, such as H⁺, NH⁴⁺, trialkylammonium, Mg²⁺, Na⁺ and the like.

Polynucleotides that are formed by 3′-5′ phosphodiester linkages are said to have 5′-ends and 3′-ends because the mononucleotides that are reacted to make the polynucleotide are joined in such a manner that the 5′ phosphate of one mononucleotide pentose ring is attached to the 3′ oxygen (i.e., hydroxyl) of its neighbor in one direction via the phosphodiester linkage. Thus, the 5′-end of a polynucleotide molecule has a free phosphate group or a hydroxyl at the 5′ position of the pentose ring of the nucleotide, while the 3′ end of the polynucleotide molecule has a free phosphate or hydroxyl group at the 3′ position of the pentose ring. Within a polynucleotide molecule, a position or sequence that is oriented 5′ relative to another position or sequence is said to be located “upstream,” while a position that is 3′ to another position is said to be “downstream.” This terminology reflects the fact that polymerases proceed and extend a polynucleotide chain in a 5′ to 3′ fashion along the template strand.

A polynucleotide may be composed entirely of deoxyribonucleotides, entirely of ribonucleotides, or chimeric mixtures thereof. Polynucleotides may be comprised of internucleotide, nucleobase and sugar analogs. Unless denoted otherwise, whenever a polynucleotide sequence is represented, it will be understood that the nucleotides are in 5′ to 3′ orientation from left to right and that “A” denotes deoxyadenosine, “C” denotes deoxycytidine, “G” denotes deoxyguanosine, and “T” denotes thymidine.

“Polynucleotide template” means the region of a polynucleotide complementary to an oligomer, probe or primer polynucleotide. It is understood that a polynucleotide template will normally constitute a portion of a larger polynucleotide molecule, with the “template” merely referring to that portion of the polynucleotide molecule to which the oligomer, probe or primer of the present invention is complementary. The term “template” thus refers to the region of the polynucleotide that constitutes the physical template for hybridization of another complementary polynucleotide. Templates may be genomic DNA, cDNA, PCR amplified DNA, or any other polynucleotide that serves as a pattern for the synthesis of a complementary polynucleotide.

“Primer” means an oligonucleotide molecule that is complementary to a portion of a target sequence and, upon hybridization to the target sequence, has a free 3′-hydroxyl group available for polymerase-catalyzed covalent bonding with a 5′-triphosphate group of a deoxyribonucleoside triphosphate molecule, thereby initiating the enzymatic polymerization of nucleotides complementary to the template. Primers may include detectable labels for use in detecting the presence of the primer or primer extension products that include the primer.

“Probe” refers to a nucleobase oligomer that is capable of forming a duplex structure by complementary base pairing with a sequence of a target polynucleotide, and further where the duplex so formed is detected, visualized, measured and/or quantitated. In some embodiments, the probe is fixed to a solid support, such as in column, a chip or other array format. Probes may include detectable labels for use in detecting the presence of the probe.

“Target”, as used in reference to a “target polymoprhic site” and the like, refer to a specific polynucleobase sequence that is the subject of hybridization with a complementary nucleobase polymer (e.g., an oligomer). The nature of the target sequence is not limiting, and can be any nucleobase polymer of any sequence, composed of, for example, DNA, RNA, substituted variants and analogs thereof, or combinations thereof. The target can be single-stranded or double-stranded. In primer extension processes, the target polynucleotide which forms a hybridization duplex with the primer may also be referred to as a “template.” A template serves as a pattern for the synthesis of a complementary polynucleotide. A target sequence for use with the present invention may be derived from any living or once living organism, including but not limited to prokaryote, eukaryote, plant, animal, and virus, as well as non-natural, synthetic and/or recombinant target sequences.

The practice of the present invention will employ, unless otherwise indicated, conventional techniques of molecular biology, microbiology, recombinant DNA techniques, and oligonucleotide synthesis which are within the skill of the art. Such techniques are explained fully in the literature. Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)); Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.), the contents of all of which are incorporated herein by reference.

Various aspects of the invention are described in further detail in the following subsections.

The present invention relates to improved methods and reagents for detection, quantification and characterization of nucleic acid templates having polymorphic variation, using binding ligands that specifically bind target protein epitopes characteristic of genetic mutations.

In one aspect, the present invention relates to a method for detecting the presence or absence of missense amino acid sequence downstream of a position corresponding to a frameshift mutation using a binding ligand, such as an antibody, that is specific for the missense amino acid sequence downstream of and resulting from the frameshift mutation. Certain frameshift mutations result in an alteration of the reading frame of a nucleic acid molecule, which may result in the nucleic acid molecule encoding different amino acids downstream of the site of the frameshift mutation, until a different stop codon is encountered. The stop codon may be created immediately after the frameshift mutation, resulting in a protein having the same amino acid sequence up to the point of the stop codon, without encoding any additional new amino acids. Alternatively, the stop codon may be created downstream of the frameshift mutation, resulting in new additional amino acid sequence between the frameshift mutation and the new stop codon that is not in the wild-type amino acid sequence. The new additional amino acid sequence between the frameshift mutation and the new stop codon that is not in the wild-type amino acid sequence is referred to herein as “missense amino acid sequence,” since it is amino acid sequence that is the direct product of a missense mutation. In accordance with the present invention, the resulting new additional amino acid sequence between the frameshift mutation and the stop codon may be unique protein sequence, which may be used to identify and characterize the protein that is the product of the frameshift mutation.

In some embodiments, the methods of the present invention provide a binding ligand that is capable of specifically binding to the missense amino acid sequence. The presence of the missense amino acid sequence can be determined by detecting binding of the binding ligand to the missense amino acid sequence, using any one of various methods available and known to those skilled in the art. Generally, the methods of the invention comprise:

(a) providing a biological sample containing a target protein encoded by a gene characterized by a missense frameshift mutation, wherein a gene having the frameshift mutation encodes a target protein comprising missense amino acid sequence downstream of the position corresponding to the frameshift mutation and a gene not having the frameshift mutation encodes a target protein comprising wild-type amino acid sequence downstream of the position corresponding to the frameshift mutation;

(b) combining with the biological sample a binding ligand capable of specifically binding the missense amino acid sequence; and

(c) determining whether the binding ligand binds to the missense amino acid sequence, wherein binding of the ligand to the missense amino acid sequence is indicative of the presence of the frameshift mutation, and the absence of binding of the ligand to the missense amino acid sequence is indicative of the absence of the frameshift mutation.

Binding Ligands Specific for Wild-Type Amino Acid Sequences

In another aspect, the present invention is directed to methods for detecting the presence or absence of a target protein having a frameshift mutation in a biological sample by determining whether a binding ligand, such as an antibody, binds to the wild-type amino acid sequence of a target protein that is downstream of the position corresponding to the frameshift mutation. The presence of the wild-type protein may in some circumstances constitute inferential evidence of the absence of a frameshift mutation. Alternatively, the absence of the wild-type protein may in some circumstances constitute inferential evidence of the presence of a frameshift mutation. In some circumstances, it is also possible that a biological sample may contain more than one allele of a gene—one having a frameshift mutation, and another not having a frameshift mutation. In such circumstances, it may be useful to detect the presence or absence of the wild-type protein, either alone or in conjunction with an independent assay to detect the presence or absence of the frameshift protein. Although detection of the presence or absence of the wild-type protein may not constitute definitive proof of the absence or presence of the frameshift mutation, in some circumstances such evidence may be sufficient for diagnostic use, particularly if it is known that only two alleles are present in a given population and individuals are capable of possessing only one of the alleles. In such cases, the presence of one allele may be sufficiently reliable evidence of the absence of the other allele.

Accordingly, in some embodiments the methods of the present invention comprise:

(a) providing a biological sample containing a target protein encoded by a gene characterized by a missense frameshift mutation, wherein a gene having the frameshift mutation encodes a target protein comprising missense amino acid sequence downstream of the position corresponding to the frameshift mutation, and wherein a gene not having the frameshift mutation encodes a target protein comprising wild-type amino acid sequence downstream of the position corresponding to the frameshift mutation;

(b) combining with the biological sample a binding ligand capable of specifically binding the wild-type amino acid sequence; and

(c) determining whether the binding ligand binds to the wild-type amino acid sequence, wherein binding of the ligand to the wild-type amino acid sequence is indicative of the absence of the missense amino acid sequence, and the absence of binding of the ligand to the wild-type amino acid sequence is indicative of the presence of the missense amino acid sequence.

In another aspect, the present invention is directed to methods for detecting the presence or absence of a nonsense frameshift mutation. A nonsense frameshift mutation is a mutation that results in a stop codon immediately after the frameshift mutation and encodes a target protein that is truncated at the position of the frameshift mutation, with no additional amino acid sequence (i.e., missense amino acid sequence) downstream of the mutation that is characteristic of the frameshift mutation. In the absence of any missense amino acid sequence that can be detected to determine the presence or absence of the frameshift mutation, the present invention employs the strategy of detecting the wild-type amino acid sequence downstream of the frameshift mutation, the presence of which may be indicative of the absence of the frameshift mutation and the absence of which may be indicative of the presence of the frameshift mutation.

Accordingly, in one particular embodiment, the invention is directed to methods for detecting the presence or absence of a target protein in a biological sample, comprising:

(a) providing a biological sample containing a target protein encoded by a gene characterized by a nonsense frameshift mutation, wherein a gene having the nonsense frameshift mutation encodes a target protein truncated at the position of the frameshift mutation, and wherein a gene not having the nonsense frameshift mutation encodes a target protein comprising wild-type amino acid sequence downstream of the position corresponding to the frameshift mutation;

(b) combining with the biological sample a binding ligand capable of specifically binding the wild-type amino acid sequence; and

(c) determining whether the binding ligand binds to the wild-type amino acid sequence, wherein binding of the ligand to the wild-type amino acid sequence is indicative of the absence of the nonsense mutation, and the absence of binding of the ligand to the wild-type amino acid sequence is indicative of the presence of the nonsense mutation.

Binding Ligands Specific for Splice-Site Amino Acid Sequences

In yet another aspect, the present invention is directed to methods for detecting the presence or absence of a protein encoded by a gene having a splice-site mutation. A splice-site mutation is a mutation that deletes an internal portion of a protein, resulting in a protein having only wild-type amino acid sequence, but having a unique splice-junction at the point where the amino acid sequences flanking the deleted portion are joined. Generally, a splice-site mutation (which results in the deletion of an internal portion of a protein, while retaining the flanking portions of the protein) will result from the deletion of only whole codon units in a gene, leaving the remaining whole codon units that encode wild-type amino acids intact. In accordance with the present invention, binding ligands capable of specifically binding the unique splice-junction may be used to detect the presence or absence of the splice-junction, which may be indicative of the presence or absence of the mutation causing the deletion.

In one particular embodiment, the invention is directed to methods for detecting the presence or absence of a target protein in a biological sample, comprising:

(a) providing a biological sample containing a target protein encoded by a gene characterized by a splice-site mutation, wherein a gene having the splice-site mutation encodes a target protein comprising wild-type sequence having a modified splice-site amino acid sequence and a gene not having the splice-site mutation encodes a target protein comprising normal wild-type amino acid sequence;

(b) combining with the biological sample a binding ligand capable of specifically binding the splice-site amino acid sequence; and

(c) determining whether the binding ligand binds to the splice-site amino acid sequence, wherein binding of the ligand to the splice-site amino acid sequence is indicative of the presence of the splice-site mutation, and the absence of binding of the ligand to the splice-site amino acid sequence is indicative of the absence of the splice-site mutation.

Use of Multiple Binding Ligands Specific for Mutation and Wild-Type Sequences

In yet another aspect, the present invention relates to methods for detecting the presence or absence of both the mutation (i.e., missense or splice-site) amino acid sequence and the wild-type amino acid sequence. Thus, in some embodiments, the methods of the present invention contemplate use of a second binding ligand specific for the wild-type amino acid sequence encoded by a wild-type gene (i.e., a gene that does not have the frameshift mutation) downstream of the position of the frameshift mutation. Because a gene not having the frameshift mutation will encode a target protein comprising wild-type amino acid sequence downstream of the position of the frameshift mutation, a second binding ligand may be used to confirm the presence (or absence) of the wild-type amino acid sequence. Similarly, one of the two binding ligands may be specific for a splice-site amino acid sequence. The second binding ligand specific for the wild-type amino acid sequence may be used alone, as described above, or alternatively the second binding ligand may be used in combination with the binding ligand specific for the mutation amino acid sequence. Used in combination, the two different binding ligands—one specific for the mutation amino acid sequence (indicative of a frameshift mutation or a deletion mutation) and another specific for the wild-type amino acid sequence (indicative of the absence of the mutation sequence)—may be useful, for example, to provide a control assay, or to test biological samples that may contain both the wild-type and mutation proteins. Thus, the methods of the invention may further comprise combining with the biological sample a second binding ligand capable of specifically binding the wild-type amino acid sequence, and determining whether the second binding ligand binds to the wild-type amino acid sequence, wherein binding of the second binding ligand to the wild-type amino acid sequence is indicative of the presence of the wild-type gene.

Use of Control Binding Ligand

In some embodiments, the methods of the invention also contemplate use of a control binding ligand specific for a region of the target protein unaffected by the frameshift mutation (e.g., upstream of the position of the frameshift mutation), together with a binding ligand specific for the missense amino acid sequence (or specific for the wild-type sequence in the absence of the frameshift mutation), to confirm the presence of the target protein, or with a binding ligand specific for the wild-type amino acid sequence of a protein no affected by a frameshift mutation. Thus, the methods of the invention may further comprise combining with the biological sample a control binding ligand capable of specifically binding the target protein upstream of the position corresponding to the frameshift mutation, and determining whether the control binding ligand binds to the target protein, wherein binding of the control binding ligand to target protein is indicative of the presence of the target protein in the sample.

In one aspect of this embodiment, the gene not having the frameshift mutation encodes a target protein comprising wild-type amino acid sequence downstream of the position corresponding to the frameshift mutation. In this aspect, the method further comprises combining with the biological sample a second binding ligand capable of specifically binding the wild-type amino acid sequence, and determining whether the second binding ligand binds to the wild-type amino acid sequence, wherein binding of the second binding ligand to the wild-type amino acid sequence is indicative of the presence of the wild-type gene. In another aspect of this embodiment, a control binding ligand is used together with a binding ligand for the missense sequence and a binding ligand for the wild-type sequence to confirm the presence of the target protein.

Antibodies

In yet another aspect, the present invention is also directed to antibodies, such as monoclonal antibodies, capable of binding capable of specifically binding a missense amino acid sequence of a target protein, wherein the missense amino acid sequence is downstream of a position corresponding to a frameshift mutation of a gene encoding the target protein. In still another aspect, the present invention is directed to antibodies capable of specifically binding a splice-site amino acid sequence of a target protein.

Production of Antibodies

The present invention is exemplified by antibodies or antigen-binding regions thereof that bind to specified epitopes of target proteins encoded by genes characterized by frameshift mutations.

Antibodies with such properties can be readily identified by one or more or a combination of the receptor competition, ELISA, co-precipitation, and/or functional assays and the crossreactivity assays described herein.

The antibodies encompassed by the present invention include IgG, IgA, IgG1-4, IgE, IgM, and IgD antibodies, e.g., IgG1κ or IgG1λ isotypes, or IgG4κ or IgG4κ isotypes. In a particular embodiment, the antibody of the present invention is a IgG2 isotype. In one embodiment, human antibodies are produced in a non-human transgenic animal, e.g., a transgenic mouse, capable of producing multiple isotypes of human antibodies to CD148 (e.g., IgG, IgA and/or IgE) by undergoing V-D-J recombination and isotype switching. Accordingly, aspects of the invention include both antibodies and antibody fragments capable of binding such specific epitopes, as well as non-human transgenic animals, B-cells, host cell transfectomas, and hybridomas which produce monoclonal antibodies. Methods of using the antibodies of the invention to detect proteins encoded by genes characterized by frameshift mutations or a related, cross-reactive receptor molecule, are also encompassed by the invention. The present invention further encompasses methods of detecting target proteins encoded by genes characterized by frameshift mutations.

The antibodies and antigen binding regions of the present invention can be constructed by any number of different methods, including, via immunization of animals (e.g., with an antigen that elicits the production of antibodies that specifically bind to and competitively inhibit the binding of at least one of an antibody of Ab-1 through Ab-8); via hybridomas (e.g., employing B-cells from transgenic or non-transgenic animals); via recombinant methods (e.g., CHO transfectomas; see, Morrison, S. (1985) Science 229:1202)), or, in vitro synthetic means (e.g., solid-phase polypeptide synthesis).

Recombinant methods for producing antibodies or antigen binding regions of the present invention begin with the isolated nucleic acid of desired regions of the immunoglobulin heavy and light chains such as those present in any of Ab-1 through Ab-8. Such regions can include, for example, all or part of the variable region of the heavy and light chains. Such regions can, in particular, include at least one of the CDRs of the heavy and/or light chains, and often, at least one CDR pair from Ab-1 through Ab-8. A nucleic acid encoding an antibody or antigen binding region of the invention can be directly synthesized by methods of in vitro oligonucleotide synthesis known in the art. Alternatively, smaller fragments can be synthesized and joined to form a larger fragment using recombinant methods known in the art. Antibody binding regions, such as for Fab or F(ab′)₂, may be prepared by cleavage of the intact protein, e.g. by protease or chemical cleavage. Alternatively, a truncated gene can be designed.

To express antibodies or antigen binding regions thereof, DNAs encoding partial or full-length light and heavy chains, can be obtained by standard molecular biology techniques (e.g., PCR amplification, site directed mutagenesis) and can be inserted into expression vectors such that the genes are operatively linked to transcriptional and translational regulatory sequences. Nucleic acids encoding an antibody or antigen binding region of the invention can be cloned into a suitable expression vector and expressed in a suitable host. A suitable vector and host cell system can allow, for example, co-expression and assembly of the variable heavy and variable light chains of at least one of Ab-1 through Ab-8, or CDR containing polypeptides thereof. Suitable systems for expression can be determined by those skilled in the art.

Nucleic acids comprising polynucleotides of the present invention can be used in transfection of a suitable mammalian or nonmammalian host cells. In some embodiments, for expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most typical because such eukaryotic cells, and in particular mammalian cells, are more likely than prokaryotic cells to assemble and secrete a properly folded and immunologically active antibody or antigen binding region.

Expression vectors include plasmids, retroviruses, cosmids, YACs, EBV derived episomes, and the like. A convenient vector is one that encodes a functionally complete human CH (constrant heavy) or CL (constant light) immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions.

The expression vector and expression control sequences are chosen to be compatible with the expression host cell used. The antibody variable heavy chain nucleic acid and the antibody variable light chain nucleic acids of the present invention can be inserted into separate vectors or, frequently, both genes are inserted into the same expression vector. The nucleic acids can be inserted into the expression vector by standard methods (e.g., ligation of complementary restriction sites on the antibody nucleic acid fragment and vector, or blunt end ligation if no restriction sites are present). The heavy and light chain variable regions of Ab-1 through Ab-8, described herein, can be used to create full-length antibody genes of any antibody isotype by inserting them into expression vectors already encoding heavy chain constant and light chain constant regions of the desired isotype (and subclass) such that the VH segment is operatively linked to the CH segment(s) within the vector and the VL segment is operatively linked to the CL segment within the vector. Additionally or alternatively, the expression vector can encode a signal peptide that facilitates secretion of the antibody or antigen binding region chain from a host cell. The antibody or antigen binding region chain gene can be cloned into the vector such that the signal peptide is linked in-frame to the amino terminus of the antibody/antigen binding region chain gene. The signal peptide can be an immunoglobulin signal peptide or a heterologous signal peptide (i.e., a signal peptide from a non-immunoglobulin protein).

In addition to the CDR comprising sequence, the expression vectors of the invention carry regulatory sequences that control the expression of the sequence in a host cell. The term “regulatory sequence” is intended to includes promoters, enhancers and other expression control elements (e.g., polyadenylation signals) that control the transcription or translation of the antibody chain genes. Such regulatory sequences are described, for example, in Goeddel; Gene Expression Technology. Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, and the like. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, nonviral regulatory sequences may be used, such as the ubiquitin promoter or beta-globin promoter.

In addition to the antibody or antigen binding region nucleic acids and regulatory sequences, the expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

Preferred mammalian host cells for expressing the recombinant antibodies or antigen binding regions of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS/0 myeloma cells, COS cells and SP2.0 cells. In particular for use with NS/0 myeloma cells, another preferred expression system is the GS gene expression system disclosed in WO 87/04462, WO 89/01036 and EP 338 841. When expression vectors of the invention are introduced into mammalian host cells, the antibodies or antigen binding regions are produced by culturing the host cells for a period of time sufficient to allow for expression of the antibody or antigen binding region in the host cells or, more preferably, secretion of the antibody or antigen binding region into the culture medium in which the host cells are grown.

Once expressed, antibodies and antigen binding regions of the invention can be purified according to standard methods in the art, including HPLC purification, fraction column chromatography, gel electrophoresis and the like (see, e.g., Scopes, Protein Purification, Springer-Verlag, NY, 1982). In certain embodiments, polypeptides are purified using chromatographic and/or electrophoretic techniques. Exemplary purification methods include, but are not limited to, precipitation with ammonium sulphate; precipitation with PEG; immunoprecipitation; heat denaturation followed by centrifugation; chromatography, including, but not limited to, affinity chromatography (e.g., Protein-A-Sepharose), ion exchange chromatography, exclusion chromatography, and reverse phase chromatography; gel filtration; hydroxylapatite chromatography; isoelectric focusing; polyacrylamide gel electrophoresis; and combinations of such and other techniques. In certain embodiments, a polypeptide is purified by fast protein liquid chromatography or by high pressure liquid chromatography (HPLC).

Generation of Hybridomas Producing Human Monoclonal Antibodies

Another aspect of the present invention includes a hybridoma cell that produces the antibody or antigen-binding region thereof of the present invention. A hybridoma cell may comprise a B cell obtained from a transgenic non-human animal having a genome comprising a human heavy chain transgene and a light chain transgene fused to an immortalized cell, wherein the hybridoma produces a detectable amount of the monoclonal antibody or antigen-binding region thereof of the present invention.

Mouse splenocytes can be isolated and fused with PEG to a mouse myeloma cell line based upon standard protocols. The resulting hybridomas are then screened for the production of antigen-specific antibodies. For example, single cell suspensions of splenic lymphocytes from immunized mice are fused to one-sixth the number of P3X63-Ag8.653 nonsecreting mouse myeloma cells (ATCC, CRL 1580) with 50% PEG. Cells are plated at approximately 2×10⁵ in flat bottom microtiter plate, followed by a two week incubation in selective medium containing 20% fetal Clone Serum, 18% “653” conditioned media, 5% origen (IGEN), 4 mM L-glutamine, 1 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After two weeks, cells are cultured in medium in which the HAT is replaced with HT. Individual wells are then screened by ELISA for human anti-CD148 monoclonal IgM and IgG antibodies. Once extensive hybridoma growth occurs, medium is observed usually after 10-14 days. The antibody secreting hybridomas are replated, screened again, and if still positive for human IgG, anti-CD148 monoclonal antibodies, can be subcloned at least twice by limiting dilution. The stable subclones are then cultured in vitro to generate small amounts of antibody in tissue culture medium for characterization.

Generation of Transfectomas Producing Human Monoclonal Antibodies

Antibodies of the invention can also be produced in a host cell transfectoma using, for example, a combination of recombinant DNA techniques and gene transfection methods as is well known in the art (Morrison, S. (1985) Science 229:1202). A transfectoma cell may comprise nucleic acids encoding a human heavy chain and a human light chain, wherein the transfectoma produces a detectable amount of the monoclonal antibody or antigen-binding region thereof of the present invention.

For example, to express the antibodies, or antibody fragments thereof, DNAs encoding partial or full-length light and heavy chains, can be obtained by standard molecular biology techniques (e.g., PCR amplification, site directed mutagenesis) and can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to includes promoters, enhancers and other expression control elements (e.g., polyadenylation signals) that control the transcription or translation of the antibody chain genes. Such regulatory sequences are described, for example, in Goeddel; Gene Expression Technology. Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, non-viral regulatory sequences may be used, such as the ubiquitin promoter or beta-globin promoter.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection). In a preferred embodiment of the present invention, the antibody chain genes and regulatory sequences are expressed in “split dhfr vectors” PDC323 and PDC324, as disclosed by Bianchi, A. A. and McGrew, J. T. (2003) “High-level expression of full antibodies using trans-complementing expression vectors,” Bioengineering and Biotechnology, 84 (4): 439-444; and McGrew, J. T. and Bianchi, A. A. (2002) “Selection of cells expressing heteromeric proteins,” U.S. patent application No. 20030082735, the contents of which are expressly incorporated herein by reference.

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS/0 myeloma cells, COS cells and SP2.0 cells. In particular for use with NS/0 myeloma cells, another preferred expression system is the GS gene expression system disclosed in WO 87/04462, WO 89/01036 and EP 338 841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Use of Partial Antibody Sequences to Express Intact Antibodies

Antibodies interact with target antigens predominantly through amino acid residues that are located in the six heavy and light chain complementarity determining regions (CDRs). For this reason, the amino acid sequences within CDRs are more diverse between individual antibodies than sequences not part of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998, Nature 332:323-327; Jones, P. et al., 1986, Nature 321:522-525; and Queen, C. et al., 1989, Proc. Natl. Acad. See. U.S.A. 86:10029-10033). Such framework sequences can be obtained from public DNA databases that include germline antibody gene sequences. These germline sequences will differ from mature antibody gene sequences because they will not include completely assembled variable genes, which are formed by V(D)J joining during B cell maturation. Germline gene sequences will also differ from the sequences of a high affinity secondary repertoire antibody at individual evenly across the variable region. For example, somatic mutations are relatively infrequent in the amino-terminal portion of framework region. For example, somatic mutations are relatively infrequent in the amino terminal portion of framework region 1 and in the carboxy-terminal portion of framework region 4. Furthermore, many somatic mutations do not significantly alter the binding properties of the antibody. For this reason, it is not necessary to obtain the entire DNA sequence of a particular antibody in order to recreate an intact recombinant antibody having binding properties similar to those of the original antibody (see PCT/US99/05535 filed on Mar. 12, 1999, which is herein incorporated by referenced for all purposes). Partial heavy and light chain sequence spanning the CDR regions is typically sufficient for this purpose. The partial sequence is used to determine which germline variable and joining gene segments contributed to the recombined antibody variable genes. The germline sequence is then used to fill in missing portions of the variable regions. Heavy and light chain leader sequences are cleaved during protein maturation and do not contribute to the properties of the final antibody. For this reason, it is necessary to use the corresponding germline leader sequence for expression constructs. To add missing sequences, cloned cDNA sequences cab be combined with synthetic oligonucleotides by ligation or PCR amplification. Alternatively, the entire variable region can be synthesized as a set of short, overlapping, oligonucleotides and combined by PCR amplification to create an entirely synthetic variable region clone. This process has certain advantages such as elimination or inclusion or particular restriction sites, or optimization of particular codons.

The nucleotide sequences of heavy and light chain transcripts from a hybridomas are used to design an overlapping set of synthetic oligonucleotides to create synthetic V sequences with identical amino acid coding capacities as the natural sequences. The synthetic heavy and kappa chain sequences can differ from the natural sequences in three ways: strings of repeated nucleotide bases are interrupted to facilitate oligonucleotide synthesis and PCR amplification; optimal translation initiation sites are incorporated according to Kozak's rules (Kozak, 1991, J. Biol. Chem. 266:19867-19870); and, HindIII sites are engineered upstream of the translation initiation sites.

For both the heavy and light chain variable regions, the optimized coding, and corresponding non-coding, strand sequences are broken down into 30-50 nucleotide approximately the midpoint of the corresponding non-coding oligonucleotide. Thus, for each chain, the oligonucleotides can be assemble into overlapping double stranded sets that span segments of 150-400 nucleotides. The pools are then used as templates to produce PCR amplification products of 150-400 nucleotides. Typically, a single variable region oligonucleotide set will be broken down into two pools which are separately amplified to generate two overlapping PCV products. These overlapping products are then combined by PCT amplification to form the complete variable region. It may also be desirable to include an overlapping fragment of the heavy or light chain constant region (including the BbsI site of the kappa light chain, or the AgeI site if the gamma heavy chain) in the PCR amplification to generate fragments that can easily be cloned into the expression vector constructs.

The reconstructed heavy and light chain variable regions are then combined with cloned promoter, translation initiation, constant region, 3′ untranslated, polyadenylation, and transcription termination, sequences to form expression vector constructs. The heavy and light chain expression constructs can be combined into a single vector, co-transfected, serially transfected, or separately transfected into host cells which are then fused to form a host cell expressing both chains.

Plasmids for use in construction of expression vectors for human IgGκ are described below. The plasmids were constructed so that PCR amplified V heavy and V kappa light chain cDNA sequences could be used to reconstruct complete heavy and light chain minigenes. These plasmids can be used to express completely human, or chimeric IgG1κ or IgG4κ antibodies. Similar plasmids can be constructed for expression of other heavy chain isotypes, or for expression of antibodies comprising lambda light chains.

Thus, in another aspect of the invention, the structural features of an antibody specific for a particular region of a target protein may be used to create structurally related antibodies that retain at least one functional property of the antibodies of the invention, such as binding to the target protein.

The ability of the antibody to bind a target protein can be determined using standard binding assays (e.g., an ELISA). Since it is well known in the art that antibody heavy and light chain CDR3 domains play a particularly important role in the binding specificity/affinity of an antibody for an antigen, the recombinant antibodies of the invention prepared as set forth above preferably comprise the heavy and light chain CDR3s of antibodies. The antibodies further can comprise the CDR2s of antibodies. The antibodies further can comprise the CDR1s of antibodies. Accordingly, the invention further provides antibodies comprising: (1) human heavy chain framework regions, a human heavy chain CDR1 region, a human heavy chain CDR2 region, and a human heavy chain CDR3 region, wherein the human heavy chain CDR3 region is the CDR3 of antibodies; and (2) human light chain framework regions, a human light chain CDR1 region, a human light chain CDR2 region, and a human light chain CDR3 region, wherein the human light chain CDR3 region is the CDR3 of antibodies, wherein the antibody binds the target protein. The antibody may further comprise the heavy chain CDR2 and/or the light chain CDR2 of antibodies. The antibody may further comprise the heavy chain CDR1 and/or the light chain CDR1 of antibodies.

Preferably, the CDR1, 2, and/or 3 of the engineered antibodies described above comprise the exact amino acid sequence(s) as the antibodies disclosed herein. However, the ordinarily skilled artisan will appreciate that some deviation from the exact CDR sequences may be possible while still retaining the ability of the antibody to bind the target protein effectively (e.g., conservative substitutions). Accordingly, in another embodiment, the engineered antibody may be composed of one or more CDRs that are, for example, 90%, 95%, 98% or 99.5% identical to one or more CDRs of antibodies.

Characterization of Binding of Monoclonal Antibodies

To characterize binding of monoclonal antibodies of the invention, sera from immunized mice can be tested, for example, by ELISA. Briefly, microtiter plates are coated with purified target protein epitopes at 0.25 μg/ml in PBS, and then blocked with 5% bovine serum albumin in PBS. Dilutions of plasma from target protein-immunized mice are added to each well and incubated for 1-2 hours at 37° C. The plates are washed with PBS/Tween and then incubated with a goat-anti-human IgG Fc-specific polyclonal reagent conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with target protein antigen. Hybridomas that bind with high affinity to the target protein will be subcloned and further characterized. One clone from each hybridoma, which retains the reactivity of the parent cells (by ELISA), can be chosen for making a 5-10 vial cell bank stored at −140° C., and for antibody purification.

To purify target protein antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C.

To determine if the selected monoclonal antibodies bind to the desired epitopes, each antibody can be biotinylated using commercially available reagents (Pierce, Rockford, Ill.). Competition studies using unlabeled monoclonal antibodies and biotinylated monoclonal antibodies can be performed using target protein coated-ELISA plates as described above. Biotinylated MAb binding can be detected with a strep-avidin-alkaline phosphatase probe.

To determine the isotype of purified antibodies, isotype ELISAs can be performed. Wells of microtiter plates can be coated with 10 g/ml of anti-human Ig overnight at 4° C. After blocking with 5% BSA, the plates are reacted with 10 g/ml of monoclonal antibodies or purified isotype controls, at ambient temperature for two hours. The wells can then be reacted with either human IgGl or human IgM-specific alkaline phosphatase-conjugated probes. Plates are developed and analyzed as described above.

In order to demonstrate binding of monoclonal antibodies to live cells expressing the target protein, flow cytometry can be used. Briefly, cell lines expressing the target protein (grown under standard growth conditions) are mixed with various concentrations of monoclonal antibodies in PBS containing 0.1% Tween 80 and 20% mouse serum, and incubated at 37° C. for 1 hour. After washing, the cells are reacted with Fluorescein-labeled anti-human IgG antibody under the same conditions as the primary antibody staining. The samples can be analyzed by FACScan instrument using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

Anti-target protein IgGs can be further tested for reactivity with target protein antigen by Western blotting. Briefly, cell extracts from cells expressing the target protein can be prepared and subjected to sodium dodecyl sulfate (SDS) polyacrylamide gel electrophoresis. After electrophoresis, the separated antigens will be transferred to nitrocellulose membranes, blocked with 20% mouse serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Bispecific/Multispecific Binding Molecules

In yet another embodiment of the invention, anti-target protein monoclonal antibodies, or antigen-binding regions thereof, can be derivatized or linked to another functional molecule, e.g., another peptide or protein (e.g., an Fab′ fragment) to generate a bispecific or multispecific molecule which binds to multiple binding sites or target epitopes, such as multiple missense sequences generated by different frameshift mutations. For example, an antibody or antigen-binding region of the invention can be functionally linked (e.g., by chemical coupling, genetic fusion, noncovalent association or otherwise) to one or more other binding molecules, such as another antibody, antibody fragment, peptide or binding mimetic.

Accordingly, the present invention includes bispecific and multispecific molecules comprising at least one first binding specificity for a target protein and a second binding specificity for a second target epitope. In a particular embodiment of the invention, the second target epitope is an Fc receptor, e.g., human FcγRI (CD64) or a human Fcα receptor (CD89). Therefore, the invention includes bispecific and multispecific molecules capable of binding both to FcγR, FcαR or FcεCR expressing effector cells (e.g., monocytes, macrophages or polymorphonuclear cells (PMNs)), and to target cells expressing the target protein. Bispecific and multispecific molecules of the invention can further include a third binding specificity, in addition to an anti-Fc binding specificity and an anti-target protein binding specificity.

In one embodiment, the bispecific and multispecific molecules of the invention comprise as a binding specificity at least one antibody, or an antibody fragment thereof, including, e.g., an Fab, Fab′, F(ab′)2, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

In other embodiments, bispecific and multispecific molecules of the invention further comprise a binding specificity which recognizes, e.g., binds to, a target cell antigen, e.g., the target protein. In one particular embodiment, the binding specificity is provided by a human monoclonal antibody of the present invention.

The antibodies which can be employed in the bispecific or multispecific molecules of the invention are murine, chimeric and humanized monoclonal antibodies.

Chimeric mouse-human monoclonal antibodies (i.e., chimeric antibodies) can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., Science 240:1041-1043 (1988); Liu et al., PNAS 84:3439-3443 (1987); Liu et al., 1987, J. Immunol. 139:3521-3526; Sun et al. (1987) PNAS 84:214-218; Nishimura et al., Canc. Res. 47:999-1005 (1987); Wood et al., Nature 314:446-449 (1985); and Shaw et al., J. Natl Cancer Inst. 80:1553-1559 (1988).

Identification of Target Protein Binding Molecules

The target protein epitopes and antibodies of the present invention may be used to identify other agents that bind the target protein epitope, which may be useful in diagnosing certain physiological disorders. In one aspect of the present invention there is provided a method for identifying a compound that specifically binds to a target protein epitope comprising: contacting a test compound with a target protein epitope for a time sufficient to form a complex and detecting for the formation of a complex by detecting the target protein epitope or the compound in the complex, so that if a complex is detected, a compound that binds to the target protein epitope is identified. For example, cells transfected with DNAs coding for proteins of interest can be treated with various drugs, and co-immunoprecipitations can be performed. Agents which may be used to bind target protein epitopes include peptides, antibodies, nucleic acids, antisense compounds or ribozymes. The nucleic acid may encode the antibody or the antisense compound. The peptide may be at least 4 amino acids of the sequence of the binding protein. Alternatively, the peptide may be from 4 to 30 amino acids (or from 8 to 20 amino acids) that is at least 75% identical to a contiguous span of amino acids of the binding protein. Agents can be tested using transfected host cells, cell lines, cell models or animals, such as described herein, by techniques well known to those of ordinary skill in the art, such as disclosed in U.S. Pat. Nos. 5,622,852 and 5,773,218, and PCT published application Nos. WO 97/27296 and WO 99/65939, each of which are incorporated herein by reference. The modulating effect of the agent can be tested in vivo or in vitro. Agents can be provided for testing in a phage display library or a combinatorial library. Exemplary of a method to screen agents is to measure the effect that the agent has on the formation of the protein complex.

The target protein epitopes of the present invention may also be used to produce structural analogs of biologically active polypeptides of interest or of small molecules with which they bind. One aspect of the present invention provides a method for identifying a compound that specifically binds to a target protein epitope comprising: providing atomic coordinates defining a three-dimensional structure of a target protein epitope, and designing or selecting compounds capable of binding the target protein epitope based on said atomic coordinates. Several approaches for use in rational drug design include analysis of three-dimensional structure, alanine scans, molecular modeling and use of anti-id antibodies. These techniques are well known to those skilled in the art. Such techniques may include providing atomic coordinates defining a three-dimensional structure of a protein complex formed by said first polypeptide and said second polypeptide, and designing or selecting compounds capable of interfering with the interaction between a first polypeptide and a second polypeptide based on said atomic coordinates.

Binding Assays

The materials and methods of the present invention are particularly useful for in vitro diagnostic assays to detect the presence or absence of a protein encoded by a gene characterized by certain mutations. Such in vitro assays are typically based on methods that involve binding of a binding ligand to a particular epitope of a target protein, and determining whether the binding ligand binds. Various types of binding assays may be used to practice the methods of the present invention, including immunological binding assays. Immunological binding assays typically utilize a capture agent to bind specifically to and often immobilize the analyte target antigen. The capture agent is a moiety that specifically binds to the analyte. In one embodiment of the present invention, the capture agent is an antibody or antigen-binding region thereof that specifically binds the target protein epitopes of the invention. These immunological binding assays are well known in the art (Asai, ed., Methods in Cell Biology, Vol. 37, Antibodies in Cell Biology, Academic Press, Inc., New York (1993)).

Immunological binding assays frequently utilize a labeling agent that will signal the presence of the bound complex formed by the capture agent and antigen. The labeling agent can be one of the molecules comprising the bound complex; i.e. it can be a labeled specific binding agent or a labeled anti-specific binding agent antibody. Alternatively, the labeling agent can be a third molecule, commonly another antibody, which binds to the bound complex. The labeling agent can be, for example, an anti-specific binding agent antibody bearing a label. The second antibody, specific for the bound complex, may lack a label, but can be bound by a fourth molecule specific to the species of antibodies which the second antibody is a member of. For example, the second antibody can be modified with a detectable moiety, such as biotin, which can then be bound by a fourth molecule, such as enzyme-labeled streptavidin. Other proteins capable of specifically binding immunoglobulin constant regions, such as protein A or protein G may also be used as the labeling agent. These binding proteins are normal constituents of the cell walls of streptococcal bacteria and exhibit a strong non-immunogenic reactivity with immunoglobulin constant regions from a variety of species. (Akerstrom, J. Immunol., 135:2589-2542 (1985); Chaubert, Mod. Pathol., 10:585-591 (1997)).

Throughout the assays, incubation and/or washing steps may be required after each combination of reagents. Incubation steps can vary from about 5 seconds to several hours, preferably from about 5 minutes to about 24 hours. However, the incubation time will depend upon the assay format, analyte, volume of solution, concentrations, and the like. Usually, the assays will be carried out at ambient temperature, although they can be conducted over a range of temperatures.

A. Non-Competitive Binding Assays

Immunological binding assays can be of the non-competitive type. These assays have an amount of captured analyte that is directly measured. For example, in one preferred “sandwich” assay, the capture agent (antibody) can be bound directly to a solid substrate where it is immobilized. These immobilized capture agents then capture (bind to) antigen present in the test sample. The protein thus immobilized is then bound to a labeling agent, such as a second antibody having a label. In another preferred “sandwich” assay, the second antibody lacks a label, but can be bound by a labeled antibody specific for antibodies of the species from which the second antibody is derived. The second antibody also can be modified with a detectable moiety, such as biotin, to which a third labeled molecule can specifically bind, such as streptavidin. (See Harlow and Lane, Antibodies, A Laboratory Manual, Ch 14, Cold Spring Harbor Laboratory, NY (1988), incorporated herein by reference).

B. Competitive Binding Assays

Immunological binding assays can be of the competitive type. The amount of analyte present in the sample is measure indirectly by measuring the amount of an added analyte displaced, or competed away, from a capture agent (antibody) by the analyte present in the sample. In one preferred competitive binding assay, a known amount of analyte, usually labeled, is added to the sample and the sample is then contacted with the capture agent. The amount of labeled analyze bound to the antibody is inversely proportional to the concentration of analyte present in the sample (See, Harlow and Lane, Antibodies, A Laboratory Manual, Ch 14, pp. 579-583, supra).

In another preferred competitive binding assay, the capture agent is immobilized on a solid substrate. The amount of protein bound to the capture agent may be determined either by measuring the amount of protein present in a protein/antibody complex, or alternatively by measuring the amount of remaining uncomplexed protein. The amount of protein may be detected by providing a labeled protein. Harlow and Lane (supra).

Yet another preferred competitive binding assay, hapten inhibition is utilized. Here, a known analyte is immobilized on a solid substrate. A known amount of antibody is added to the sample, and the sample is contacted with the immobilized analyte. The amount of antibody bound to the immobilized analyte is inversely proportional to the amount of analyte present in the sample. The amount of immobilized antibody may be detected by detecting either the immobilized fraction of antibody or the fraction that remains in solution. Detection may be direct where the antibody is labeled or indirect by the subsequent addition of a labeled moiety that specifically binds to the antibody as described above.

C. Utilization of Competitive Binding Assays

The competitive binding assays can be used for cross-reactivity determinations to permit a skilled artisan to determine if a protein or enzyme complex which is recognized by a peptibody of the invention is the desired protein and not a cross-reacting molecule or to determine whether the peptibody is specific for the antigen and does not bind unrelated antigens. In assays of this type, antigen can be immobilized to a solid support and an unknown protein mixture is added to the assay, which will compete with the binding of the peptibodies to the immobilized protein. The competing molecule also binds one or more antigens unrelated to the antigen. The ability of the proteins to compete with the binding of the peptibodies to the immobilized antigen is compared to the binding by the same protein that was immobilized to the solid support to determine the cross-reactivity of the protein mix.

D. Other Binding Assays

The present invention also provides Western blot methods to detect or quantify the presence of a CD148 epitope or fragment thereof in a sample. The technique generally comprises separating sample proteins by gel electrophoresis on the basis of molecular weight and transferring the proteins to a suitable solid support, such as nitrocellulose filter, a nylon filter, or derivatized nylon filter. The sample is incubated with antibodies or antigen-binding regions thereof that specifically bind a CD148 epitope and the resulting complex is detected. These peptibodies may be directly labeled or alternatively may be subsequently detected using labeled antibodies that specifically bind to the peptibody.

E. Diagnostic Assays

The derivative binding agents, such as peptides and peptibodies or fragments thereof, of the present invention are useful for the diagnosis of conditions or diseases characterized by expression of target protein epitopes that are indicative of particular types of mutations. Diagnostic assays for target protein epitopes include methods utilizing an antibody and a label to detect target protein epitopes in human body fluids or extracts of cells or tissues. The antibodies of the present invention can be used with or without modification. In a preferred diagnostic assay, the antibodies will be labeled by attaching, e.g., a label or a reporter molecule. A wide variety of labels and reporter molecules are known, some of which have been already described herein. In particular, the present invention is useful for diagnosis of human disease.

A variety of protocols for measuring target protein epitopes using antibodies specific for the respective protein epitope are known in the art. Examples include enzyme-linked immunosorbent assay (ELISA), radioimmunoassay (RIA) and fluorescence activated cell sorting (FACS). A multi-site, monoclonal-based immunoassay utilizing monoclonal antibodies reactive to multiple non-interfering epitopes on the target protein is preferred, but a competitive binding assay can be employed. These assays are described, for example, in Maddox et al., J. Exp. Med., 158:1211 (1983).

In order to provide a basis for diagnosis, normal or standard values for target protein expression are usually established. This determination can be accomplished by combining body fluids or cell extracts from normal subjects, preferably human, with an antibody to the target protein, under conditions suitable for complex formation that are well known in the art. The amount of standard complex formation can be quantified by comparing the binding of the antibodies to known quantities of the target protein, with both control and disease samples. Then, standard values obtained from normal samples can be compared with values obtained from samples from subjects potentially affected by disease. Deviation between standard and subject values suggests a role of the target protein mutations in the disease state.

For diagnostic applications, in certain embodiments antibodies, or antigen-binding regions thereof, of the present invention typically will be labeled with a detectable moiety. The detectable moiety can be any one that is capable of producing, either directly or indirectly, a detectable signal. For example, the detectable moiety may be a radioisotope, such as 3H, 4C, 32P, 35S, or 125I, a fluorescent or chemiluminescent compound, such as fluorescein isothiocyanate, rhodamine, or luciferin; or an enzyme, such as alkaline phosphatase, beta-galactosidase, or horseradish peroxidase. Bayer et al., Meth. Enz., 184: 138-163, (1990).

Kits

In still another aspect, the present invention is directed to kits comprising a binding ligand capable of specifically binding a missense amino acid sequence of a target protein, wherein the missense amino acid sequence is downstream of a position corresponding to a frameshift mutation of a gene encoding the target protein.

The present invention is further illustrated by the following examples of genetic mutations that may be detected using the methods and principles of the present invention. The following examples should not be construed as further limiting, and it will be understood by those skilled in the art that the methods and principles of the present invention may be applied to other genetic mutations as well. The contents of all figures and all references, patents and published patent applications cited throughout this application are expressly incorporated herein by reference.

Detecting Splice Variants in SEEK1 Gene

The methods and materials of the present invention may be utilized to detect certain splice variant mutations in the SEEK1 gene. The SEEK1 gene (GenBank ABO31479) is characterized by polymorphic variation on the 2p21.3 gene, which are associated with psoriasis in the Swedish population. Holm et al., Experimental Dermatology 12:435-444 (2003). The full-length SEEK1 gene (GenBank AF484-418) is comprised of 152 amino acids, a portion of which is reproduced below.

Exon 3 Exon 4 Exon 5 Exon 6

----|--------|--------------------------------------|---------
MTCTDQKSHSQRALGTQTPALQGPQLLNTDPSSKETRPPHVNPDRLCHMEPANHFWHAGDLQAMISKE-(SEQ ID NO:1)

A deletion mutation in the SEEK1 gene causes a splice of exon 1 and exon 6, as shown below, resulting in a truncated SEEK1 protein consisting of approximately 100 amino acids (GenBank AF484419).

Exon 1 Exon 6

------|--------------------------
MASRRHAGDLQAMISKE---(SEQ ID NO:2)

In accordance with the present invention, antibodies to all or part of the amino acid sequence encoded by Exon 3-Exon 5 shown above, as well as a portion of the amino acid sequence upstream of the exon 6 sequence HAGDLQAMISKE, can be used to detect the presence or absence of the full-length protein.

In addition, antibodies that specifically bind to the new exon 1/exon 6 splice junction (but do not bind to the full-length protein) can also be used to detect the presence of the shorter protein MASRRHAGDL (binding is indicative of a protein having the splice mutation, while failure to bind is indicative of the wild-type protein).

Detecting Stop Codon Mutations in Connexin 26 Gene

Connexin 26 is a gap junction protein (GJB2) that is important in hearing. Polymorphic variants of Connexin 26 are known to be caused by frame shift mutations that result in a truncated protein. For example, one polymorphic variant of Connexin 26 is the Del25G mutation, which results in a premature stop codon and the following prematurely truncated protein at amino acid 13:

Stop Codon
1 mdwgtlqtil ggv--------(SEQ ID NO:3)

Another significant polymorphic variant of Connexin 26 is the Del167 mutation, which also results in an altered frameshift, which introduces a stop codon and the following truncated protein at amino acid 55:

Stop codon
1 mdwgtlqtil ggvnkhstsi gkiwltvlfi frimilvvaa kevwgdeqad fvcnt-------(SEQ ID NO:4)

The methods and materials of the present invention may be used to prepare binding ligands, such as antibodies, that specifically bind the wild-type sequence of Connexin 26 downstream of the C-terminal amino acids of the Del25G and Del167 truncated proteins. Binding ligands specific for the region downstream of amino acid 55 can be used to determine the presence or absence of the full-length Connexin 26 protein (binding is indicative of the presence of the full-length protein, while failure to bind is indicative of either the Del25G or Del167 mutation). Binding ligands specific for the region defined by amino acids 14-55 may also be used (binding is indicative of the presence of either the full-length protein or the Del167 mutation, while failure to bind is indicative of either the Del25G or Del167 mutation). Control binding ligands may also be used as a control to confirm the presence or absence of any form of the Connexin 26 gene.

Detecting Frameshift Mutations in the Apolipoprotein A-1 Gene

Deficiency in the Apolipoprotein A-1 protein has been reported to be caused by a frameshift mutation resulting in a different amino acid sequence following the frame shift mutation (Yokata et al., Atherosclerosis 162:399-407(2002)). Wild-type apolipoprotein consists of 243 amino acids. A deletion of the nucleic acid C at codon 184 (Del552C) causes a frameshift that results in 16 amino acid residues of missense sequence following the frameshift and introduces a new stop codon at amino acid 200. The following diagrams illustrate the differences between the apolipoprotein having the frameshift mutation (top) and the wild-type (bottom):

(SEQ ID NO:5)
ctc aag gag aag gcg gcg cca gac tgg ccg agt acc acg cca agg cca ccg agc atc tga
L K E K A A P D W P S T T P R P P S I End AA 200
Mutant amino acid sequences (200 AA)
(SEQ ID NO:6)
ctc aag gag aac ggc ggc ggc aga ctg gcc gag tac cac gcc aag gcc acc gag cat ctg-----cag tga
L K E N G G A R L A E Y H A K A T E H L-------Q END AA 243

Monoclonal or polyclonal antibodies specific for the 16 amino acid missense sequence between the frameshift mutation and the stop codon can be generated and used to detect the presence or absence of the apolipoprotein frameshift mutation. A diagnostic test based on detection of this frameshift mutation could be of potential value in detecting apolipoprotein frameshift mutations associated with coronary heart disease.

Detecting Frameshift Mutations in Surface Protein B

It has been demonstrated that vaccination with the outer surface protein B (OspB) from Borrelia burgdorferi (Bb) strain B31 protected mice from infection with Bb B31 but not against Bb N40 (Fikrig et al., Proc Natl Acad. Sci. 90(9):4092-4096 (1993)). The Bb N40 spirochetes which evade vaccination immunity to OspB (i.e., which remain resistant to treatment) have been shown to have a truncated form of OspB, due to a TAA stop codon at nucleotide 577.

Thus, the binding ligands may be prepared according to the present invention and used to detect the presence or absence of resistant forms of OspB characterized by a frameshift mutation that causes a prematurely truncated form of OspB, which is indicative of resistant.

Detecting Frameshift Mutations in TCF7L2 Gene

A variant of the transcription factor 7-like 2 (TCF7L2) gene has been shown to be associated with risk of type 2 diabetes (Grant et al., Nature Genetics., 2006, published online 15 January; doi:1038/ng1732). Changes in the 3′ end of the TCF7L2 gene can result in a large number of protein isoforms with short, medium, and long COOH-terminal ends in various colorectal cancer cell lines (Duval et al., Cancer Research 60:3872-3879 (Jul. 15, 2000)).

These changes can be detected by peptide binding molecules as described above.

Detecting Frameshift Mutations in CBP Protein

A fraction of lung cancer cell lines exhibited mutations and/or deletions in the cyclic AMP response element binding protein-binding protein (CBP) (Kishimoto et al., Clinical Cancer Research 11:512-519 (2005)). A role for this protein in cancer has been suggested by previous functional and genetic studies.

Detecting Frameshift Mutations Characteristic of Human Esophageal Cancer

Gene alterations of the cyclic AMP response element binding protein binding protein (CBP), a nuclear transcriptional coactivator protein in esophageal squamous cell carcinoma samples, have been shown to result in modified protein sequence. (So et al., Clinical Cancer Research 11:19-27 (2004)). Many of the genetic alterations were in the regions encoding the histone acetyltransferase domain and COOH-terminal transactiving domain one of the CBP gene.

Detecting Frameshift Mutations Characteristic of Prostate Cancer

Alpha-methylacyl-CoA racemase (AMACR) is a protein that is overexpressed in prostate cancer. Alternate splice variants of AMACR have been reported in exon 5, which cause a deletion of 749 bp and a resulting shift in the reading frame (Mubiru et al., The Prostate 65(2): 117-123 (2005)). The resulting protein product has a different molecular weight and isoelectric point than the native wild-type protein. Moreover, the COOH end of the variant protein does not contain the peroxisomal targeting signal found in the native protein.

What is claimed is:

1. A method for detecting the presence or absence of a target protein in a biological sample, comprising:
providing a biological sample containing a target protein encoded by a gene characterized by a missense frameshift mutation, wherein a gene having the frameshift mutation encodes a target protein comprising missense amino acid sequence downstream of the position corresponding to the frameshift mutation and a gene not having the frameshift mutation encodes a target protein comprising wild-type amino acid sequence downstream of the position corresponding to the frameshift mutation;
combining with the biological sample a binding ligand capable of specifically binding the missense amino acid sequence; and
determining whether the binding ligand binds to the missense amino acid sequence, wherein binding of the ligand to the missense amino acid sequence is indicative of the presence of the frameshift mutation, and the absence of binding of the ligand to the missense amino acid sequence is indicative of the absence of the frameshift mutation.

2. The method of claim 1, wherein the binding ligand is a monoclonal antibody.

3. The method of claim 1, further comprising:
combining with the biological sample a control binding ligand capable of specifically binding the target protein upstream of the position corresponding to the frameshift mutation; and
determining whether the control binding ligand binds to the target protein, wherein binding of the control binding ligand to target protein is indicative of the presence of the target protein in the sample.

4. The method of claim 3, wherein the control binding ligand is a monoclonal antibody.

5. The method of claim 1, further comprising:
combining with the biological sample a second binding ligand capable of specifically binding the wild-type amino acid sequence; and
determining whether the second binding ligand binds to the wild-type amino acid sequence, wherein binding of the second binding ligand to the wild-type amino acid sequence is indicative of the presence of the wild-type gene.

6. The method of claim 5, wherein the second binding ligand is a monoclonal antibody.

7. The method of claim 5, further comprising:
combining with the biological sample a control binding ligand capable of specifically binding the target protein upstream of the position corresponding to the frameshift mutation; and
determining whether the control binding ligand binds to the target protein, wherein binding of the control binding ligand to target protein is indicative of the presence of the target protein in the sample.

8. The method of claim 7, wherein the control binding ligand is a monoclonal antibody.

9. A method for detecting the presence or absence of a target protein in a biological sample, comprising:
providing a biological sample containing a target protein encoded by a gene characterized by a missense frameshift mutation, wherein a gene having the frameshift mutation encodes a target protein comprising missense amino acid sequence downstream of the position corresponding to the frameshift mutation, and wherein a gene not having the frameshift mutation encodes a target protein comprising wild-type amino acid sequence downstream of the position corresponding to the frameshift mutation;
combining with the biological sample a binding ligand capable of specifically binding the wild-type amino acid sequence; and
determining whether the binding ligand binds to the wild-type amino acid sequence, wherein binding of the ligand to the wild-type amino acid sequence is indicative of the absence of the missense amino acid sequence, and the absence of binding of the ligand to the wild-type amino acid sequence is indicative of the presence of the missense amino acid sequence.

10. The method of claim 9, wherein the binding ligand is a monoclonal antibody.

11. The method of claim 9, further comprising:
combining with the biological sample a control binding ligand capable of specifically binding the target protein upstream of the position corresponding to the frameshift mutation; and
determining whether the control binding ligand binds to the target protein, wherein binding of the control binding ligand to target protein is indicative of the presence of the target protein in the sample.

12. The method of claim 11, wherein the control binding ligand is a monoclonal antibody.

13. The method of claim 9, further comprising:
combining with the biological sample a second binding ligand capable of specifically binding the wild-type amino acid sequence; and
determining whether the second binding ligand binds to the wild-type amino acid sequence, wherein binding of the second binding ligand to the wild-type amino acid sequence is indicative of the presence of the wild-type gene.

14. The method of claim 13, wherein the second binding ligand is a monoclonal antibody.

15. The method of claim 13, further comprising:
combining with the biological sample a control binding ligand capable of specifically binding the target protein upstream of the position corresponding to the frameshift mutation; and
determining whether the control binding ligand binds to the target protein, wherein binding of the control binding ligand to target protein is indicative of the presence of the target protein in the sample.

16. The method of claim 15, wherein the control binding ligand is a monoclonal antibody.

17. A method for detecting the presence or absence of a target protein in a biological sample, comprising:
providing a biological sample containing a target protein encoded by a gene characterized by a nonsense frameshift mutation, wherein a gene having the nonsense frameshift mutation encodes a target protein truncated at the position of the frameshift mutation, and wherein a gene not having the nonsense frameshift mutation encodes a target protein comprising wild-type amino acid sequence downstream of the position corresponding to the frameshift mutation;
combining with the biological sample a binding ligand capable of specifically binding the wild-type amino acid sequence; and
determining whether the binding ligand binds to the wild-type amino acid sequence, wherein binding of the ligand to the wild-type amino acid sequence is indicative of the absence of the nonsense mutation, and the absence of binding of the ligand to the wild-type amino acid sequence is indicative of the presence of the nonsense mutation.

18. The method of claim 17, wherein the binding ligand is a monoclonal antibody.

19. The method of claim 17, further comprising:
combining with the biological sample a control binding ligand capable of specifically binding the target protein upstream of the position corresponding to the frameshift mutation; and
determining whether the control binding ligand binds to the target protein, wherein binding of the control binding ligand to target protein is indicative of the presence of the target protein in the sample.

20. The method of claim 19, wherein the control binding ligand is a monoclonal antibody.

21. A method for detecting the presence or absence of a target protein in a biological sample, comprising:
providing a biological sample containing a target protein encoded by a gene characterized by a splice-site mutation, wherein a gene having the splice-site mutation encodes a target protein comprising wild-type sequence having a modified splice-site amino acid sequence and a gene not having the splice-site mutation encodes a target protein comprising normal wild-type amino acid sequence inserted within the splice-site amino acid sequence, such that the splice site amino acid sequence is modified;
combining with the biological sample a binding ligand capable of specifically binding the splice-site amino acid sequence; and
determining whether the binding ligand binds to the splice-site amino acid sequence, wherein binding of the ligand to the splice-site amino acid sequence is indicative of the presence of the splice-site mutation, and the absence of binding of the ligand to the splice-site amino acid sequence is indicative of the absence of the splice-site mutation.

22. The method of claim 21, wherein the binding ligand is a monoclonal antibody.

23. The method of claim 21, further comprising:
combining with the biological sample a control binding ligand capable of specifically binding the target protein upstream of the position corresponding to the splice-site mutation; and
determining whether the control binding ligand binds to the target protein, wherein binding of the control binding ligand to target protein is indicative of the presence of the target protein in the sample.

24. The method of claim 21, wherein the control binding ligand is a monoclonal antibody.

25. The method of claim 24, further comprising:
combining with the biological sample a second binding ligand capable of specifically binding the wild-type amino acid sequence inserted within the splice-site amino acid sequence; and
determining whether the second binding ligand binds to the wild-type amino acid sequence, wherein binding of the second binding ligand to the wild-type amino acid sequence inserted within the splice-site amino acid sequence is indicative of the absence of the splice-site mutation.

26. The method of claim 21, wherein the second binding ligand is a monoclonal antibody.

27. The method of claim 25, further comprising:
combining with the biological sample a control binding ligand capable of specifically binding the target protein upstream of the position corresponding to the splice-site mutation; and
determining whether the control binding ligand binds to the target protein, wherein binding of the control binding ligand to target protein is indicative of the presence of the target protein in the sample.

28. The method of claim 21, wherein the control binding ligand is a monoclonal antibody.

29. An antibody capable of specifically binding a missense amino acid sequence of a target protein, wherein the missense amino acid sequence is downstream of a position corresponding to a frameshift mutation of a gene encoding the target protein.

30. An antibody according to claim 29, wherein the antibody is a monoclonal antibody.

31. An antibody capable of specifically binding a splice-site amino acid sequence of a target protein.

32. An antibody according to claim 31, wherein the antibody is a monoclonal antibody.

33. A kit comprising a binding ligand capable of specifically binding a missense amino acid sequence of a target protein, wherein the missense amino acid sequence is downstream of a position corresponding to a frameshift mutation of a gene encoding the target protein.

34. A kit according to claim 33, wherein the binding ligand is a monoclonal antibody.

35. A kit according to claim 33, further comprising a control binding ligand capable of specifically binding wild-type amino acid sequence of a target protein, wherein the wild-type amino acid sequence is upstream of the position corresponding to the frameshift mutation.

36. A kit according to claim 35, wherein the control binding ligand is a monoclonal antibody.

37. A kit comprising a binding ligand capable of specifically binding a splice-site amino acid sequence of a target protein.

38. A kit according to claim 37, wherein the binding ligand is a monoclonal antibody.

39. A kit according to claim 37, further comprising a control binding ligand capable of specifically binding wild-type amino acid sequence of a target protein, wherein the wild-type amino acid sequence is upstream of the position corresponding to the splice-site mutation.

40. A kit according to claim 39, wherein the control binding ligand is a monoclonal antibody.
